(19) United States
(12) Patent Application Publication
(10) Pub. No.: US 2014/0155498 A1
(43) Pub. Date: Jun. 5, 2014

(54) Method of Manufacture of Stable Liquid Coagulation Factors

(71) Applicant: Leon Wortham, Chattanooga, TN (US)

(72) Inventor: Leon Wortham, Chattanooga, TN (US)

(21) Appl. No.: 14/175,077

(22) Filed: Feb. 7, 2014

Related U.S. Application Data
(63) Continuation-in-part of application No. 13/651,152, filed on Oct. 12, 2012, now abandoned.

(51) Int. Cl. A61K 47/36

(52) U.S. Cl. CPC A61K 47/36
USPC 514/778; 436/69

(57) ABSTRACT

A diagnostic or therapeutic formulation of stable liquid coagulation factors comprises at least one coagulation factor, at least one stabilizer, and optional additives. The stabilizer is either amylopectin, galatomannan polysaccharide, agarose, agaropectin, and gelatin, or a blend of amylopectin, galatomannan polysaccharide, agarose, agaropectin, and gelatin. A preferred additive is the zwitterionic buffer 3-[N-tris(hydroxymethyl)methylamino)-2-hydroxypropane sulfonic acid (TAPSO). The stable liquid coagulation factors can be used as reagents and controls in plasma clotting assays. In addition the therapeutic formulation can be used for factor replacement therapy. The stable liquid clotting factors are made by combining the coagulation factor(s), stabilizer(s), and additive(s) in a liquid carrier and sterilizing the mixture. Coagulation factors can be concentrated by selective precipitation from plasma. In addition, the clotting activity of the formulation can be adjusted by adding a factor concentrate to a less concentrated formulation.

The present application is a continuation in part of U.S. patent application Ser. No. 13/651,152 filed Oct. 12, 2012

### BACKGROUND

Bleeding, coagulation, and clot dissolution are regulated by a complex system of interrelated pathways collectively known as hemostasis. Impairments of the hemostatic system can be either inherited or acquired. Congenital defects of individual coagulation factors are associated with classic hemophilia A and other bleeding disorders, such as Christmas disease, PTA deficiency, Hageman trait, and hypoprothrombinemia. Acquired coagulation disorders can involve deficiencies of multiple coagulation factors. They are often associated with trauma, liver disease, vitamin K deficiency, or anticoagulant therapy. Management of these disorders requires an accurate diagnosis of the underlying hemostatic impairment and, in some cases, replacement therapy involving transfusions of blood, plasma, or antihemophilic concentrates.

The hemostatic mechanism consists of a series of activation stages in which circulating coagulation factors are converted in sequence from inactive precursors to activated forms. Activation of the hemostatic mechanism is specific for each of the two activation pathways in clot formation: the extrinsic and the intrinsic systems. In the extrinsic system, factor VII is activated by tissue thromboplastin and forms a complex by binding to factor VII and phospholipid. Calcium ions are introduced and the inactive factor X is transformed into the active factor Xa. The intrinsic pathway is activated by the binding of factor XII to subendothelial collagen. Activated factor XII forms a complex with factor XI, thus activating it to XIa. Factor XIa then activates factor IX, which forms a new complex with factor VIII, phospholipids and calcium ions. The latter complex activates factor X to Xa.

After the activation of factor X both the extrinsic and intrinsic systems merge together and follow a common pathway to clot formation. Factor Xa forms a complex (prothrombin-converting complex) with factor V, phospholipids and calcium ions, which then activates prothrombin to thrombin. The latter is a proteolytic enzyme, not normally present in plasma, that converts fibrinogen into soluble fibrin monomer. In the course of this conversion, fibrinopeptides A and B are released. Fibrin monomers polymerize spontaneously in the presence of calcium to form a soluble fibrin clot.

Impairments of the extrinsic and intrinsic pathways are generally diagnosed with laboratory tests that measure clotting time. Such measurements occur by introducing reagents into plasma samples that artificially trigger the clotting system. The partial thromboplastin time (PTT) test, which is initiated by adding cephaloplastin, calcium ions and ellagic acid or magnesium-aluminum silicates, screens the intrinsic pathway of the coagulation system. The PTT tests for the adequacy of factors XII, high-molecular-weight kininogen (HMWK), prekallikren (PK), XI, IX, and VIII. The prothrombin time (PT) test, which is triggered by thromboplastin and calcium, screens the extrinsic or tissue factor-dependent pathway and tests for the adequacy of factor VII. Both the PTT and PT tests also evaluate the common coagulation pathway involving factors II, V, and X. A third clotting assay, the thrombin time (TT), screens for fibrinogen abnormalities that prevent the formation of fibrin clots.

The biological activity of diagnostic and therapeutic formulations containing coagulation factors rests on a number of conditions, such as the original source of the coagulation factors; the method of extracting the factor(s); and the final composition, which may include buffers, salts, and stabilizers. Most of the prepared diagnostic formulations on the market today are only available as lyophilized materials, primarily for reasons of control and reagent stability. Reconstituted lyophilized diagnostic formulations have a shelf life of approximately six to thirty-six hours. Moreover, the limited stability of some coagulation factors, particularly Factors V and VII, can impact the effectiveness of plasma, factor concentrates, and related therapeutic agents used in factor replacement therapy.

The stability of coagulation factors within diagnostic and therapeutic formulations is important to the clinician, or user, as they are expensive and the longer the shelf fife, the less formulation that must be discarded due to expiration. Furthermore, there are inherent problems associated with a lyophilized product that are either reduced or eliminated in a liquid product. These include quantitation errors, the introduction of impure water, and microbial contamination during reconstitution. In addition, a lyophilized product is inherently more turbid than a liquid formulation. Reducing turbidity is particularly desirable in a diagnostic formulation, since clot detection is the end point of diagnostic assays, like the PT and PTT assays.

What is needed is a stable liquid formulation of coagulation factors that is ready to use, i.e., pre-mixed, pre-measured, and pre-sterilized, which retains full potency for extended periods, thereby assuring a long shelf-life. In terms of convenience, stability, reliability, and effectiveness, the use of such stable liquid formulations for diagnostic assays and factor replacement therapy would be of value to clinicians, patients, and researchers.

### SUMMARY

The present invention satisfies the need for ready-to-use hemostatic agents with long shelf lives. In particular, this invention relates to a diagnostic or therapeutic formulation for the detection or treatment of clotting disorders. More particularly, the invention is directed to compositions of matter that include certain stabilizers to prevent the formulations from losing their biological activity during storage. In another aspect it relates to a method of preparing the stable diagnostic or therapeutic formulation. In yet another aspect it relates to diagnostic and therapeutic processes.

The composition of matter comprises a coagulation factor and a stabilizer in a liquid carrier. The coagulation factor is a component in a biochemical pathway involved in clot formation. It can be a protein, enzyme, co-enzyme, lipoprotein, or phospholipid. Moreover, the coagulation factor can be a blood product or a recombinant protein. In addition, the coagulation factor can be a plurality of coagulation factors, such as the factors found in normal human plasma or in plasmas deficient in Factors II, V, VII, VIII, IX, X, XI, XII, XIII, high molecular weight kininogen, plasminogen activator inhibitor, t-plasminogen activator, prekallikrein, protein C, protein S, and protein X.

The stabilizer is present in an amount that retards the degradation of the coagulation factor(s) when the composition of matter is refrigerated at 2° C. to 8° C. It can be amylopectin, galatomannan polysaccharide, agarose, agaropectin, and gelatin or a blend of amylopectin, galatomannan polysaccharide, agarose, agaropectin, and gelatin preferably present in an amount of from 0.001 to 500 grams per liter of the composition.

The composition of matter may also include a zwitterionic buffer present in an amount from about 0.001 to about 1.0 moles per liter. A preferred buffering compound is 3-[N-tris(hydroxymethyl)methylamino]-2-hydroxypropanesulfonic acid (TAPSO), which is included to maintain the pH within a range of about 6 to 9. A preferred pH range is about 7 to 8 and most preferred pH for the composition of matter is about 7.4.

When the composition of matter is for use as a control in clotting assays, at least ten coagulation factors that provide essential components of the extrinsic or intrinsic clotting system are present, in amounts of about 1 unit of each factor per ml of the control.

When the composition of matter is for use as a factor deficient control in clotting assays, at least nine coagulation factors that provide essential components of the extrinsic or intrinsic clotting system are present, in amounts of about 1 unit of each factor per ml of the control, while at least one factor is lacking.

When the composition of matter is a single factor control, the coagulation factor can be fibrinogen, thrombin, proteins C, S, or X present in an amount that effectively promotes or inhibits a clotting reaction.

When the composition is a reagent that activates the extrinsic or intrinsic clotting system, the reagent can include phospholipid, thromboplastin, calcium, cephaloplastin, ellagic acid, and/or magnesium aluminum silicates.

A composition intended for use as a therapeutic agent in factor replacement therapy should be aseptic and can include one or more coagulation factors present in an amount that ensures clot formation when administered intravenously.

The composition of matter is prepared by combining the stabilizer, zwitterionic buffer, and coagulation factor(s) in a liquid carrier, adjusting the pH of the solution to between about 6 and 9, and sterilizing the formulation.

A factor concentrate can be prepared by a series of selective precipitation steps. A liquid carrier containing at least one coagulation factor is mixed with barium chloride in an amount sufficient to form a first precipitate and a first supernatant. The first precipitate is then isolated from the first supernatant. The first precipitate is then resuspended in a solution of ammonium sulfate to form a second precipitate and a second supernatant. The second supernatant is separated from the second precipitate and the concentration of ammonium sulfate in the second supernatant is increased to form a third precipitate. After collecting the third precipitate, it is resuspended in a dialysis buffer that contains liquid carrier, sorbitol or gum stabilizer, and at least one additive selected from the group consisting of a zwitterionic buffer, an antibiotic agent, and sodium citrate. The resuspended third precipitate is held within a dialysis membrane capable of retaining the coagulation factor and dialyzed against the dialysis buffer.

A coagulation control can be prepared by combining the stabilizer, zwitterionic buffer, and plasma to form a buffered solution. The buffered solution is then tested in a clotting assay. If needed, the buffered solution is adjusted by adding factor concentrate in an amount sufficient to bring the results of the clotting assay within a desired range.

Individual diagnostic formulations can be dispensed into one or more containers and assembled into a test kit for use in a clotting assay. For example, a test kit may comprise one container with an activating reagent and another container with a control.

An assay method, utilizing the diagnostic formulations described above, includes the steps of combining a plasma sample and a stabilized liquid reagent, introducing calcium ions to initiate clot formation, and measuring the time it takes for a clot to form. The assay can further comprise adding a stabilized factor deficient plasma with the sample and reagent to pinpoint a single factor deficiency. In addition, an assay method can include a step combining a stabilized control with a stabilized reagent and subsequently comparing the sample clotting time with the control clotting time.

Further, a stable liquid formulation of coagulation factor can be used for factor replacement therapy in vivo by intravenous infusion of an effective dose of the therapeutic formulation.

### DRAWINGS

These and other features, aspects, and advantages of the present invention win become better understood with regard to the following description, appended claims, and accompanying drawings where:

FIG. 1 is a graph of the effect of TAPSO concentration on the activity of normal plasma in a Prothrombin Time assay.

FIG. 2 is a graph of the effect of TAPSO concentration on the activity of normal plasma in a Partial Thromboplastin Time assay.

FIG. 3A is a graph of the effect of amylopectin concentration on the activity of a Level I control plasma in a Prothrombin Time assay.

FIG. 3B is a graph of the effect of amylopectin concentration on the activity of Level I control plasma in a Partial Thromboplastin Time assay.

FIG. 4A is a graph of the effect of amylopectin concentration on the activity of a Level II control plasma in a Prothrombin Time assay.

FIG. 4B is a graph of the effect of amylopectin concentration on the activity of Level II control plasma in a Partial Thromboplastin Time assay.

FIG. 5A is a graph of the effect of amylopectin concentration on the activity of a Level III control plasma in a Prothrombin Time assay.

FIG. 5B is a graph of the effect of amylopectin concentration on the activity of Level II control plasma in a Partial Thromboplastin Time assay.

### DESCRIPTION

The present invention is based, in part, on the surprising discovery that certain compounds stabilize a diagnostic or therapeutic formulation containing one or more factors involved in hemostasis. The liquid diagnostic or therapeutic formulation is stable for at least one month up to more than six months at about 2° to 8° C.

A “stable” composition is one that retains at least 90% of its original biological activity. The percent of activity can be determined by preparing a standard curve from dilutions of starting factor(s). For a clotting assay, the clotting time is then plotted versus the percent of factor activity. Once this curve is established, time in seconds can be converted to percent activity and a range of clotting times can be established that represents 90 to 100 percent of the factor's activity. If the composition of matter, when first produced, provides a clotting assay with one unit or 100% of activity, the “stable” composition will provide at least 0.9 units or 90% of activity when re-tested at a later time.

The liquid formulations are aqueous solutions containing coagulation factor(s), stabilizer (amylopectin, galatomannan polysaccharide, agarose, agaropectin, or a blend of amylopectin, galatomannan polysaccharide, agarose, agaropectin, and or gelatin), and optionally zwitterionic buffer (TAPSO), antibiotic agent, and/or albumin. Typical and preferred concentrations of the components are shown in Table 1:

TABLE 1
FINAL FORMULATION OF STABLE
LIQUID COAGULATION FACTORS

| | CONCENTRATION | | | | | |
|---|---|---|---|---|---|---|
| COMPO- | | | | | Most | |
| NENT | Typical | | Preferred | | Preferred | |
| Coagulation | ≧1 | unit/ml | 1-100 | units/ml | 1 | unit/ml |
| factor(s) | | | | | | |
| Stabilizer | 0.001-500 | g/l | 30-250 | g/l | 45-200 | f/l |

| Buffer | 0.001-1.0M | | 0.03-0.08M | 0.05M | |
|---|---|---|---|---|---|
| Antibiotic | 0-1 | g/l | — | 1 | g/l |
| Agent | | | | | |
| Albumin | 0-100 | g/l | — | 100 | g/l |

Useful diagnostic and therapeutic formulations can be prepared by simply mixing the ingredients, adjusting the pH, and sterilizing the solution by filtration. Moreover, a concentrated formulation of factors can be prepared from clarified plasma by a series of selective precipitation steps, followed by resuspending the final precipitate in a solution containing stabilizer(s) and additive(s). The clotting activity of a formulation, which may have become diluted by the addition of stabilizer(s) and additive(s), can be adjusted by the addition of factor concentrate.

For storage purposes, the formulations are dispensed into sterile containers. Formulations for use in diagnostic assays can be assembled as test kits of two or more containers. These test kits provide a convenient assortment of stable liquid diagnostic formulations that can be used directly as reagents and/or controls in assays for hemostatic activity.

A commonly performed clotting assay that can utilize the diagnostic formulations is the prothrombin time (PT) test. A standard curve is prepared with dilutions of stabilized normal plasma controls and stabilized thromboplastin can be used as an activating reagent. Another common clotting assay is the partial thromboplastin time (PTT) assay, which employs a partial thromboplastin reagent for activation. Both assays measure the time interval between the addition of an activating reagent and the appearance of a clot in citrated plasma sample.

Factor activity in an in vitro clotting assay, such as the PT or PTT tests, provides strong evidence that a therapeutic formulation is suitable for use in clinical transfusion practice. Consequently, another version of the present invention is to administer an effective dose of the stable liquid coagulation factor by intravenous infusion.

### DETAILED DESCRIPTION

The present invention is conveniently prepared, stored, and utilized in liquid form. The liquid carrier can be any aqueous solution or biological fluid that retains the solubility of the coagulation factors, stabilizers, and other additives of the composition. Pharmaceutically acceptable solutions are chosen for formulations intended for in vivo administration. Typical liquid carriers are plasma, water, and saline.

The coagulation factors are components of hemostatic mechanisms, including proteins, enzymes, co-enzymes, lipoproteins, and phospholipids from the extrinsic, intrinsic, and fibrinolytic pathways. The plasma fraction of blood provides a natural source of most coagulation factors, including Factors I, II, V, VII, VIII, IX, X, XI, XII, and XIII. In addition, phospholipid containing preparations of tissue thromboplastin (Factor III) and partial thromboplastin (cepahaloplastin) can be derived from tissue extracts. Consequently, individual factors can be purified from plasma, tissue extracts, or, alternatively, from recombinant DNA expression systems. In general, one unit of a coagulation factor is the amount of that factor found in 1 ml of normal plasma, whereas a factor concentrate contains greater than one unit up to about 100 units of factor per ml.

Various formulations of plasmas, coagulation factor combinations, and single coagulation factors are useful as controls in diagnostic assays for hemostatic disorders. Normal human plasma contains at least ten coagulation factors that are integral components of the intrinsic and extrinsic pathways. Activation of these normal plasma components initiates a series of reactions resulting in the production of a detectible product, usually a fibrin clot. The rate of production of the detectible product is directly proportional to the concentration of key factors in the hemostatic pathways.

A normal plasma control can be used for gauging the extrinsic or intrinsic clotting system. A normal control generally contains at least ten integral factors needed to actuate clot formation through the extrinsic and intrinsic pathways. Each coagulation factor is present in an amount that does not limit the rate of clot formation. Accordingly, a concentration of about 1 unit of each factor per ml. of plasma is preferred.

Additional controls can be formulated that deviate from normal plasma in prescribed ways. For example, Level I, II, and III controls are commonly formulated to give normal, somewhat abnormal, and decidedly abnormal clotting times in diagnostic assays. In addition, plasmas deficient in one or more coagulation factors are useful in pinpointing which individual factors may be lacking in a sample exhibiting a hemostatic impairment. Deficient plasmas can be selected from the group consisting of Factor II deficient, Factors II and VII deficient, Factors II, VII, and X deficient, Factor V deficient, Factor VII deficient, Factors VII and X deficient, Factor VIII deficient, Factor IX deficient, Factor X deficient, Factor XI deficient, Factor XII deficient Factor XIII deficient, high molecular weight kininogen deficient, plasminogen activator inhibitor deficient, t-plasminogen activator deficient, prekallikrein deficient, protein C deficient, protein S, and protein X deficient plasmas.

Single factor controls, such as fibrinogen, thrombin, proteins C, S, and XI can be prepared in a similar manner. If the single coagulation factor promotes clot formation, the control can provide an amount that is not rate-limiting. Conversely, if the single factor is an inhibitor of coagulation, the control can provide an inhibitory amount of the factor.

Other useful diagnostic formulations include activation reagents containing thromboplastin, cephaloplastin, phospholipid, calcium, ellagic acid and/or magnesium silicate particles. A reagent containing thromboplastin along with calcium can activate the extrinsic clotting system. Alternatively, an active partial thromboplastin reagent that contains cephaloplastin, and ellagic acid or magnesium-aluminum silicates, can trigger the intrinsic pathway when combined with calcium ions. These coagulation factors are specific for their respective clotting pathways and are present in an amount that is not rate-limiting.

Useful therapeutic formulations can include single factor concentrates that contain greater than 1 unit of coagulation factor per ml of the composition. They may also include stabilized plasma or other pharmaceutically acceptable fluids that contain a plurality of coagulation factors. The therapeutic formulations are substantially aseptic to ensure the safety of in vivo administrations.

The diagnostic and therapeutic formulations of the present invention are stabilized by a combination of techniques. Of primary importance is the addition of sorbitol or gum stabilizer to the composition. Factors V and VIII have particularly high rates of degradation. Sorbitol or gum retards this process. Factors I, II, VII, IX, X, XI, XII, XIII and phospholipids, while not as unstable as factors V and VIII, also undergo degradation over time. This process is also retarded by sorbitol or gum stabilizer. Accordingly, the stabilizer is maintained in the diagnostic and therapeutic formulations in an amount sufficient to retard the degradation of the coagulation factors. The galatomannan polysaccharide (gum) stabilizer can belong to the family of natural gums, for example, a preferred stabilizer is acacia gum. The gum is typically present in an amount of about 0.001 to 150 grams per liter of formulation. A preferred concentration range of gum is from 30 to 75 grams per liter, and a most preferred range is from 45 to about 60 grams per liter of the formulation. Similarly, the concentration of amylopectin is typically in the range of 0.001 to about 100 grams per liter. A preferred concentration range is about 10 to 25 grams per liter and a most preferred concentration of stabilizer is about 15 to 20 grams per liter of formulation. Gum concentrations below about 0.001 grams per liter or amylopectin concentrations below about 0.001 grams per liter, exhibit no improved stability for diagnostic and therapeutic formulations. Moreover, gum concentrations above about 150 grams per liter or sorbitol concentrations above about 100 grams per liter provide no further increase in stability, while the viscosity of the composition becomes undesirably high.

The coagulation factors can be further stabilized by the inclusion of one or more additives, such as zwitterionic buffer, sodium citrate, albumin, and antibiotic agents. For example, the addition of a zwitterionic buffer, preferably 3-[N-tris(hydroxymethyl) methylamino]-2-hydroxypropane sulfonic acid (TAPSO), has a beneficial effect on the stability of the preparation. The zwitterionic buffer is typically present in an amount of about 0.001 to about 1.0 moles per liter. A preferred concentration range is 0.03 to 0.08 moles per liter. A most preferred concentration of buffer is about 0.05 moles per liter. The buffer typically provides a pH range of from 6 to 9, a preferred pH range of 7 to 8, and an optimal pH of about 7.4. The presence of a zwitterionic buffer in an amount above 1.0 moles per liter is not preferred as such concentrations create too high an ionic strength, which destabilizes the coagulation factors. Concentrations below about 0.001 moles per liter are likewise avoided because the beneficial effect of the zwitterionic buffer is diminished.

A proposed mechanism for the stabilizing effects of sorbitol, gum, albumin, and zwitterionic buffers is that these components associate with the coagulation factors. This process blocks the association of other detrimental components, which may degrade the coagulations factors. In addition, the sorbitol, gum, and zwitterionic buffers may also prevent deactivation by maintaining an effective three-dimensional conformation of the coagulation factors. The diagnostic and therapeutic formulations can be prepared by combining liquid carrier, at least one coagulation factor, and zwitterionic buffer to form a buffered solution. The liquid carrier and coagulation factors are typically in the form of plasma. The stabilizer is mixed with the buffered solution and the pH is adjusted, within the range of 6 to 9, by the addition of acid or base as needed.

A concentrated formulation of at least one coagulation factor can also be prepared by a series of selective precipitation steps. If necessary, the starting material, which contains coagulation factor in liquid carrier, can be passed through a sieve to remove solid particles. Barium chloride is added to the starting material in an amount sufficient to form a first precipitate. A suitable amount of barium chloride is about 0.1 volumes of 1M barium chloride per liter of starting material. The first precipitate is then isolated from the first supernatant, and resuspended in a solution of ammonium sulfate. The concentration of the ammonium sulfate solution is generally less than about 50% weight per volume and preferably about 40% weight per volume of solution. The resulting second precipitate is separated from the second supernatant, typically by centrifugation. The concentration of ammonium sulfate in the second supernatant is then increased, typically to more than 50% weight per volume of second supernatant. A preferred concentration of ammonium sulfate is about 60% weight per volume of second supernatant. A third precipitate is then collected and resuspended in dialysis buffer, which contains at least one stabilizer and at least one additive. The resuspended third precipitate is then placed within a dialysis membrane having a pore size sufficiently small to retain the coagulation factor, and dialyzed against the dialysis buffer.

A method for adjusting coagulation controls to give the desired results in a clotting time assay begins by testing the activity of a starting material, which contains at least one coagulation factor, in a clotting time assay. The starting material is then combined with at least one stabilizer and zwitterionic buffer to form a buffered solution and re-tested. If the added ingredients have the effect of lengthening the clotting time, sufficient factor concentrate is added to the buffered solution to bring the clotting time back within a desired range.

The stabilizers and additives of the present invention are typically contaminated by microorganisms. Accordingly, the diagnostic and therapeutic formulations are sterilized, preferably by passing the composition through a filter sufficiently small to remove the microorganisms. Alternatively, each ingredient, e.g. liquid carrier, coagulation factor(s), sorbitol, gum, zwitterionic buffer, albumin, or antibiotic agent, may be filtered as separate solutions. A preferred filter has a pore size no greater than about 0.2 microns, which is sufficient to remove bacteria.

In addition to sterilizing the composition, it is important that there be no contamination with bacteria from other sources. Accordingly the water, containers, and equipment used in preparing the composition should be substantially free of microorganisms. Moreover, the addition of antibiotic agents, such as sodium azide, can inhibit the growth of microbial contaminants introduced before, during, or after preparing the composition.

Stable liquid diagnostic formulations can be used directly in clotting assays, such as the PT and PTT. In a typical assay aliquots of sample plasma and/or controls, thromboplastin or partial thromboplastin reagent, and calcium salt are combined. Clot formation can be detected by a variety of methods, such as the manual tilt and loop methods, or automated techniques using turbidimetric, fibrometric and photo-optic technologies.

In vitro clotting assays are predictive of a therapeutic formulation's ability to carry out clot formation in vivo. Consequently, the stable liquid coagulation factors of the present invention can also serve as a therapeutic agent in factor replacement therapy. An effective regimen of replacement therapy is one that reduces the time for clot formation in vivo. A suitable method for administering an effective dose of coagulation factor is by intravenous infusion. Therefore, the stable liquid coagulation factors of the present invention can be infused intravenously for the purpose of factor replacement therapy. A preferred method of administration entails the use of factor concentrate, which contains at least one unit of activity per milliliter of concentrate.

The previously described versions of the present invention have many advantages, including convenience, consistency, and accuracy of results. Moreover, the extended shelf life of the present invention prevents wasteful disposal of outdated formulations.

### EXAMPLES

### Example 1

### Stability of Normal Plasma Controls Containing TAPSO

This example demonstrates that select amounts of the zwitterionic buffer (TAPSO) can help retain the clotting activity in normal plasma. Solid TAPSO was added to normal human citrated plasma to give concentrations ranging from zero to 100 millimoles per liter. The standards were stored at 4°, 25°, 37°, and 41° C. for 0, 1, 2, 3, 4, and 5 days.

Prothrombin Time assays were performed on a daily basis according to the following procedure:

Reagents and standards were pre-warmed for about 3 minutes at 37° C.

A 0.1 ml aliquot of the TAPSO containing plasma was pipetted into a tube.

Timing began when a 0.2 ml of a reagent containing thromboplastin with calcium was added to the reagent and incubated at 37° C.

The mixture was monitored photo-optically. Timing automatically stopped when optical density readings dropped below a predetermined threshold level indicating clot formation.

Partial Thromboplastin Time assays were likewise performed on a daily basis according to the following procedure:

1. Reagents and standards were pre-warmed for about 3 minutes at 37° C.

2. A 0.1 ml aliquot of the TAPSO containing plasma was pipetted into a tube.

3. 0.1 ml of a reagent containing cephaloplastin and ellagic acid was added to the to the plasma and the mixture was maintained at 37° C. for about 3 minutes.

4. Timing began when a 0.1 ml aliquot of 20 mM calcium chloride was added and the mixture was incubated at 37° C.

Timing was automatically stopped when clot formation was detected as described above.

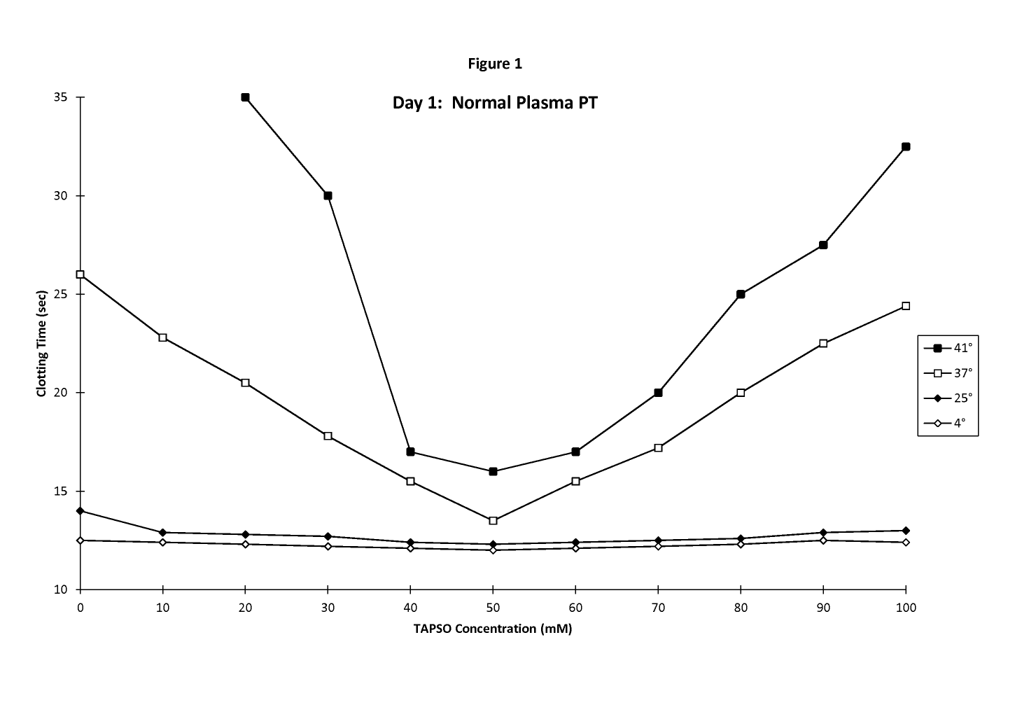

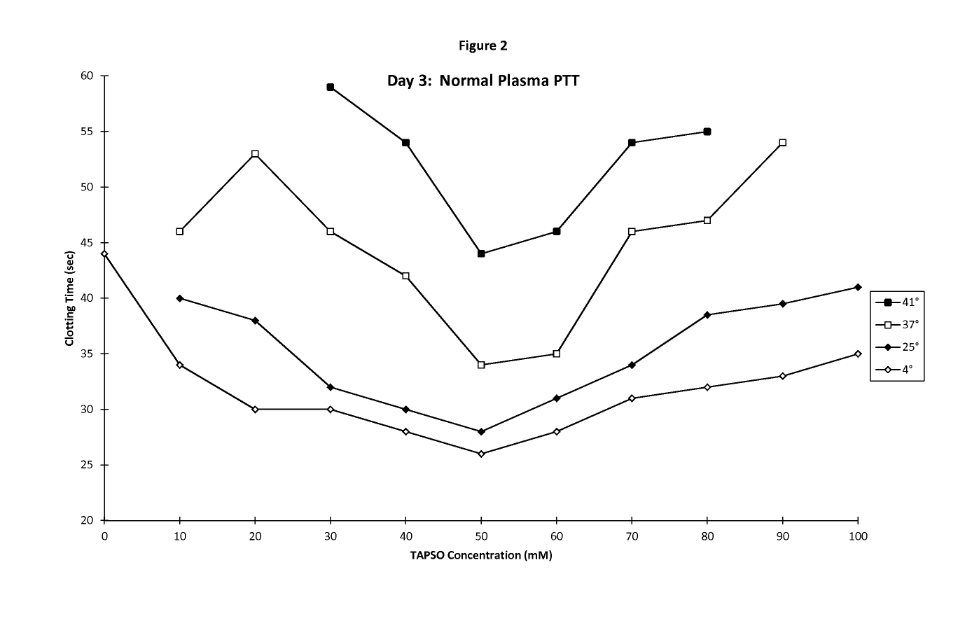

Typical results of the Prothrombin Time and Partial Thromboplastin Time assays are shown diagrammatically in FIGS. 1 and 2, respectively. Clotting times for both assays indicated that the degradation rate for clotting activity in normal plasma was reduced with respect to storage time and elevated temperature when TAPSO was present in the plasma. Although the presence of any TAPSO slowed degradation somewhat, an optimal TAPSO concentration was evident at about 50 mM.

### Example 2

### Stability of Level I, II, and III Plasma Standards Containing Amylopectin

This example demonstrates that the presence of the stabilizer, amylopectin, reduces the degradation of clotting activity in plasma controls. Solid amylopectin was added to Levels I, II, and III plasma standards in amounts ranging from zero to 300 grams per liter. In addition, sufficient TAPSO was added to all standards to give a concentration of 50 mM. These Level I, II and III standards are generally used to establish normal, somewhat abnormal, and decidedly abnormal clotting times, respectively. Results from PT clotting assays using Level I controls are considered normal within the range of about 10 to 14 seconds, and for PTT tests the normal range is generally about 20 to 38 seconds. Level II clotting times are slightly elevated, having PT results of about 15 to 20 seconds and PTT results of about 40 to 54 seconds. Level III clotting times are abnormally elevated, with PT results of about 20 to 35 seconds and PTT results of about 55 to 90 seconds. The standards were stored at 4°, 25°, 37°, and 41° C. for 0, 1, 2, 3, 4, and 5 days.

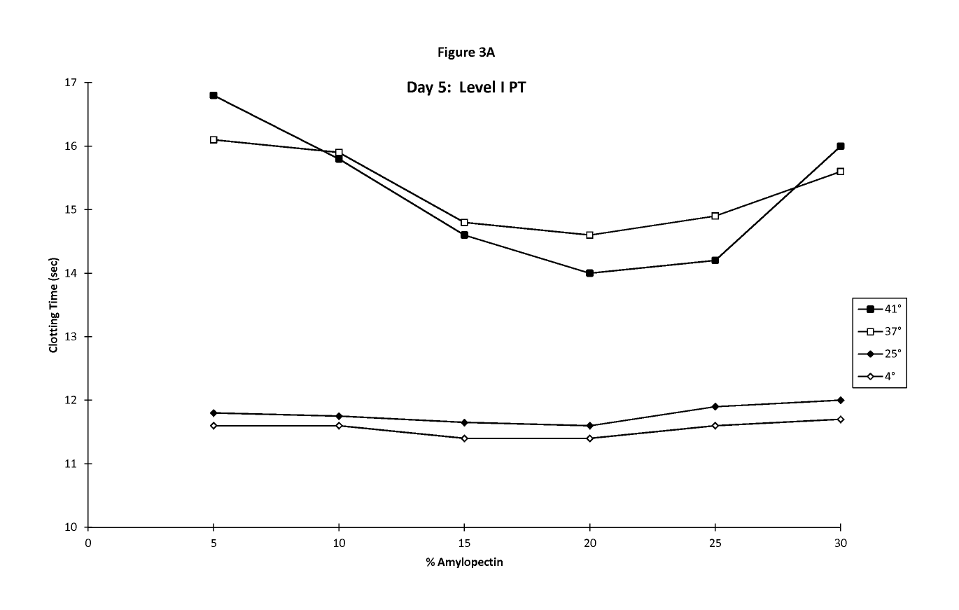

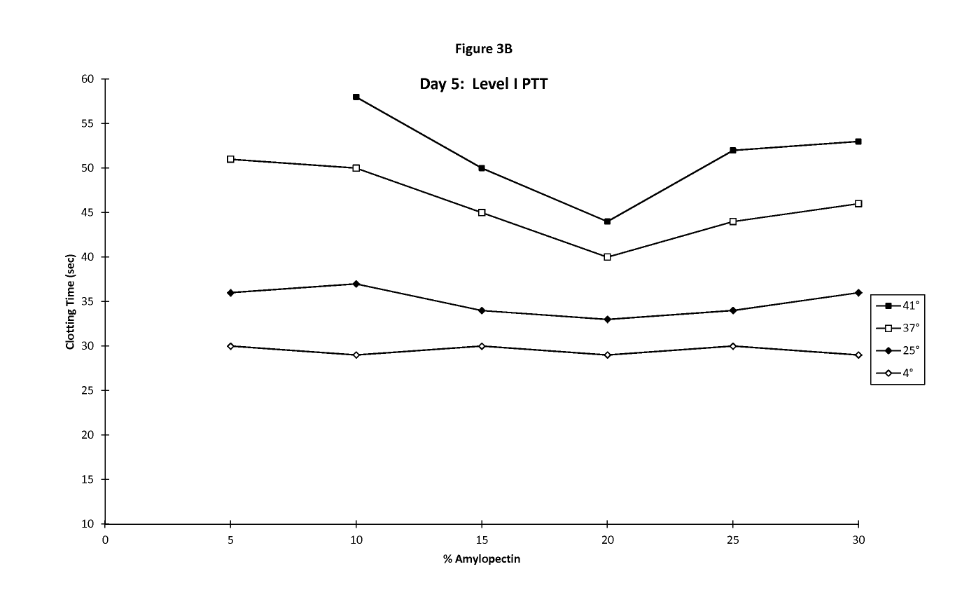

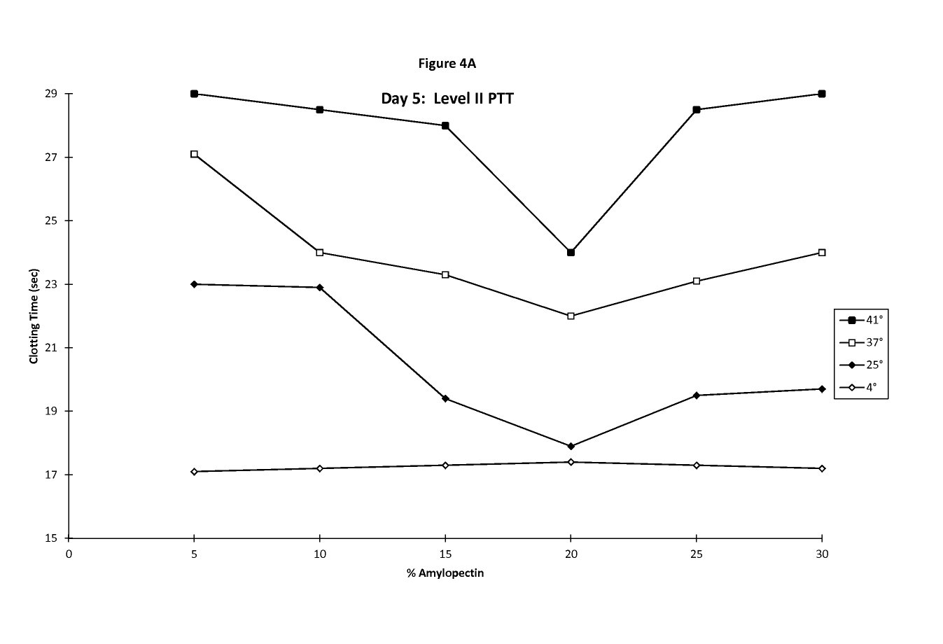

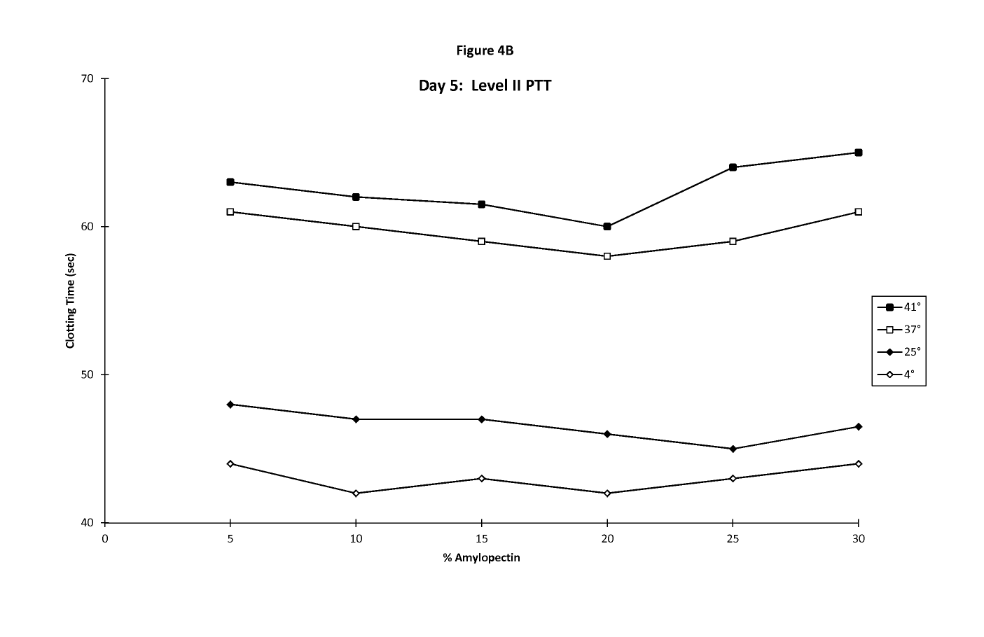

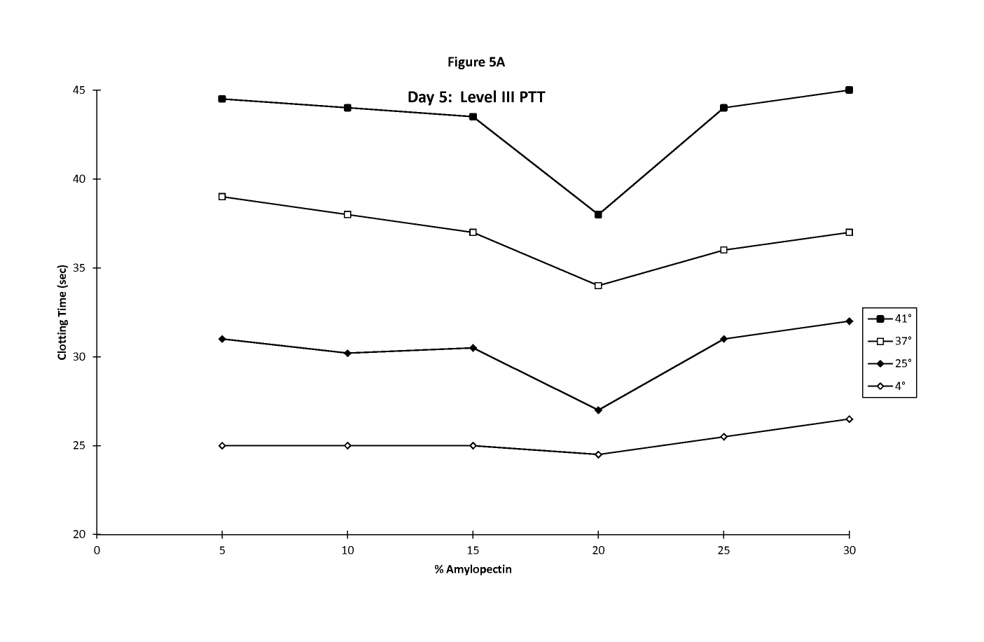

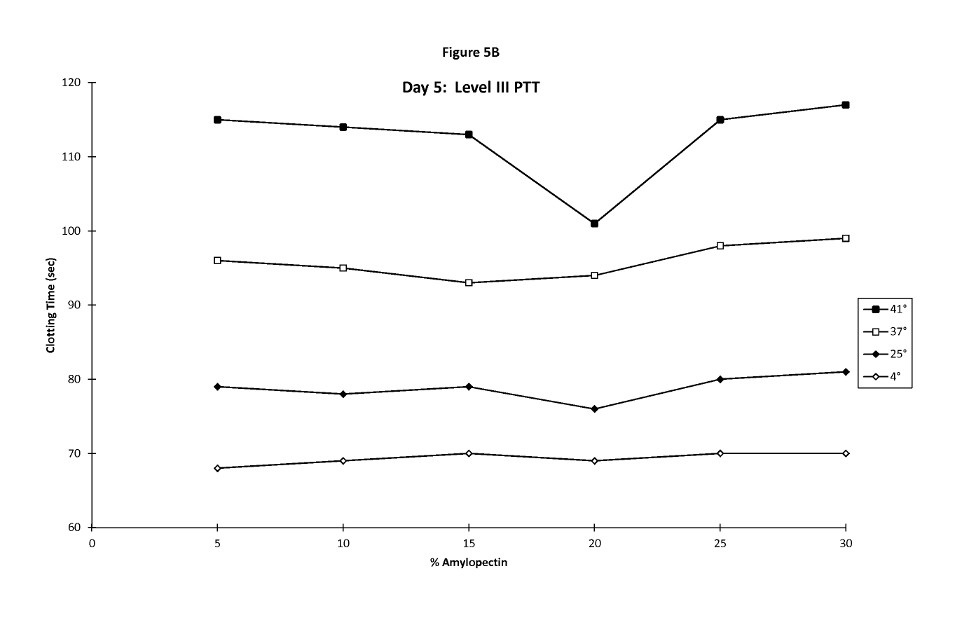

Prothrombin Time and Partial Thromboplastin Time assays were performed on a daily basis according to the procedures presented in the previous example. Results of the Prothrombin Time and Partial Thromboplastin Time assays from Day 5 are shown in FIGS. 3, 4 and 5, for Level I, II, and III, respectively. The clotting activity of the controls in both types of assay was better preserved when amylopectin was present in the plasma. An optimal concentration for minimizing the degradation of clotting activity was about 20% (wt/vol) amylopectin.

Example 3

### Method of Preparing Stable Liquid Plasma

This example shows haw to prepare a stabilized composition of matter using plasma, TAPSO, amylopectin or gum, and sodium azide as starting materials. Solid TAPSO, in an amount of 12.965 grams, was added to one liter of aseptically collected plasma. While mixing the buffer and plasma combination, the following components were added: 200 grams of amylopectin and 1.0 gram of sodium azide.

Alternatively, a gum concentrate was prepared by dissolving 300 grams of acacia gum per liter of a 3.8% citrate solution. In place of the amylopectin, 150 ml of the 30% gum, 3.8% citrate solution was mixed with one liter of TAPSO-buffered plasma.

The mixtures were stirred far about 10 minutes, and then the pH was measured. The pH was adjusted, as needed, to 7.4 with stack solutions of 5 N sodium hydroxide or 5 N hydrochloric acid. The compositions were then filtered through autoclaved 0.2 micron filters into 1-liter reagent battles, which were steam sterilized with ethylene oxide.

### Example 4

### Method of Preparing a Stable Liquid Factor Concentrate

This example shows how to stabilize coagulation factors that have been concentrated from plasma. Bovine citrated plasma was passed through two to three layers of cheesecloth and pooled in a plastic container, which had been rinsed in 3.8% sodium citrate. While mixing moderately at 2 to 8° C., 1 M BaCL₂ was added in an amount equivalent to 0.1 volumes of the total plasma volume. After mixing for one hour, the mixture was centrifuged for 10 minutes at 5,000 rpm at 2 to 8° C. The supernatant was discarded and the precipitate was resuspended in 40% (NH₄)₂SO₄ in an amount equivalent to one tenth the starting volume of plasma. After mixing for 12 to 16 hours at 2 to 8° C., the suspension was centrifuged at 5,000 rpm for 10 min at 2 to 8° C. The precipitate was discarded and the supernatant was retained. Solid (NH₄)₂S0₄ was added to the supernatant in an amount sufficient to raise the concentration to 60% (NH₄)₂S0₄. The suspension was mixed for 2 to 3 hours at 2 to 8° C. and the precipitate was centrifuged at 12,000 rpm for 30 minutes at 2 to 8° C. After discarding the supernatant the precipitate was resuspended in a small volume of dialysis solution comprising 50 mM TAPSO, 3.8% sodium citrate, 0.1% sodium azide, and 20% sorbitol at pH 7.4. The resuspended precipitate was placed in dialysis tubing, having a pore size that retains compounds with a molecular weight greater than 10,000, and dialyzed for 12 to 16 hours at 2 to 8° C. against the dialysis solution described above. Any precipitate remaining after the dialysis step was removed by centrifugation at 5,000 rpm for 10 minutes at 2 to 8° C.

### Example 5

### Real Time Stability Studies

This example demonstrates the stability of coagulation controls that were stored under refrigeration for up to 12 months. Three production lots for each of Level I, II, and III control plasmas were prepared essentially as described in Example 3. The PT and PTT values for each lot was determined as described in Example 1. The clotting times for each lot was brought back within normal range by adding approximately 5 ml of a factor concentrate, prepared according to Example 4, per liter of stabilized control plasma. The stabilized Level I, II, and III coagulation controls were stored up to 1 month at 2° C. to 8° C. in plastic screwcap containers. At one week intervals, the extrinsic and intrinsic clotting systems were tested by PT and PTT assays, respectively. Assays were conducted in duplicate, as described for Example 1, using Baxter-Dade (Dade, Fla.) reagents and an MLA 800 Instrument (Medical Laboratories Analyzer, Pleasantville, N.Y.) as a coagulometer. The results, shown in Table 2, show that the clotting times of all three lots remained within acceptable limits for at least six months.

TABLE 2
OPEN VIAL STABILITY STUDY

| | | PT (seconds) | | | | | PTT (seconds) | | | | |
|---|---|---|---|---|---|---|---|---|---|---|---|
| 2-8° C. | WEEK | 0 | 1 | 2 | 3 | 4 | 0 | 1 | 2 | 3 | 4 |
| Lot #1 | Level I | 11.2 | 12.1 | 13 | 14.0 | 14.9 | 23 | 26 | 29 | 32 | 35 |
| | Level II | 16 | 17.4 | 18.5 | 19.9 | 20.3 | 38 | 42 | 46 | 50 | 55 |
| | Level III | 22.1 | 25.2 | 28.2 | 31.3 | 34.3 | 57 | 62 | 67 | 72 | 77 |
| Lot #2 | Level I | 10.8 | 11.7 | 12.7 | 13.6 | 14.5 | 22 | 25 | 28 | 31 | 34 |
| | Level II | 16.4 | 17.8 | 19.2 | 20.7 | 22.1 | 39 | 43 | 47 | 51 | 55 |
| | Level III | 22.4 | 25.5 | 28.6 | 31.8 | 33.9 | 58 | 62 | 67 | 71 | 75 |
| Lot #3 | Level I | 10.4 | 11.4 | 12.4 | 13.3 | 14.3 | 25 | 28 | 31 | 34 | 37 |
| | Level II | 15.8 | 17.3 | 18.7 | 19.9 | 21.4 | 37 | 41 | 45 | 49 | 53 |
| | Level III | 23 | 26.2 | 29.5 | 32.7 | 35.9 | 58 | 62 | 66 | 71 | 75 |
| | | PT (seconds) | | | | | PTT (seconds) | | | | |
| −10° C. | MONTH | 0 | 1 | 2 | 3 | 4 | 0 | 1 | 2 | 3 | 4 |
| Lot #1 | Level I | 11.2 | 11.1 | 11 | 11.2 | 11.1 | 23 | 23 | 23 | 23 | 23 |
| | Level II | 16 | 16.2 | 15.9 | 16 | 16 | 38 | 38 | 38 | 38 | 38 |
| | Level III | 22.1 | 21.8 | 22 | 22.1 | 22 | 57 | 57 | 57 | 57 | 57 |
| Lot #2 | Level I | 10.8 | 10.7 | 10.8 | 10.8 | 10.7 | 22 | 22 | 22 | 22 | 22 |
| | Level II | 16.4 | 16.5 | 16.3 | 16.3 | 39 | 39 | 39 | 39 | 39 | 39 |
| | Level III | 22.4 | 22.0 | 22.3 | 22.1 | 22.3 | 58 | 58 | 58 | 58 | 58 |
| Lot #3 | Level I | 10.4 | 10.4 | 10.2 | 10.3 | 10.4 | 25 | 25 | 25 | 25 | 25 |
| | Level II | 15.8 | 15.9 | 15.5 | 15.8 | 15.6 | 37 | 37 | 37 | 37 | 37 |
| | Level III | 23 | 22.8 | 23.1 | 22.7 | 23.1 | 58 | 58 | 58 | 58 | 58 |

The average rate of degradation was determined as the change in clotting time per day at 2-8° C., and change in clotting time per 3 months for each level of control at −10° C. The results for the PT and PTT tests are shown in Table 3.

TABLE 3

| | |
|---|---|
| PT | PTT |

AVERAGE RATE OF DEGRADATION (Δseconds/day)

| | | |
|---|---|---|
| 2-8° C. | | |
| Level I | 0.01095 | 0.0331 |
| Level II | 0.01592 | 0.0442 |
| Level III | 0.0348 | 0.0485 |

AVERAGE RATE OF DEGRADATION (Δseconds/3 months)

| | | |
|---|---|---|
| −10° C | | |
| Level I | 0.0 | 0.0 |
| Level II | 0.0 | 0.0 |
| Level III | 0.0 | 0.0 |

These rates of degradation are indicative of controls that can remain within standard range, for 30 days.

The stabilized Level I, II, and III coagulation controls that were stored at −10° C. in plastic screwcap containers, the extrinsic and intrinsic clotting system, at 3 month intervals, the PT and PTT assayed in duplicate shown in Table 2, show that the clotting time for all three lots remained the same for timeline months after repetitive freeze-thaw cycles.

Although the present invention has been described in considerable detail with reference to certain preferred versions thereof, other versions are possible. For example the diagnostic formulations can be used as reagents and/or controls in an immunologically based assay for hemostatic disorders. Therefore, the spirit and scope of the appended claims should not be limited to the description of the preferred versions contained herein.

What is claimed is:

1. A method for preparing a composition of matter, comprising:
(a) combining at least one stabilizer, a zwitterionic buffer and at least one coagulation factor in a liquid carrier to form a buffered solution, wherein the coagulation factor is a component in a cascade pathway that leads to clot formation and the stabilizer is selected from the group consisting of amylopectin, galatomannan polysaccharide, agarose, agaropectin, and gelatin, present in an amount sufficient to retard the degradation of the coagulation factor for at least one month when the composition of matter is stored at 2° C. to 8° C.;
(b) adjusting the pH of the solution to between about 6 to about 9; and
(c) sterilizing the solution.

2. A method for preparing a composition of matter containing at least one coagulation factor, comprising the steps of:
(a) mixing a liquid containing at least one coagulation factor with barium chloride in an amount sufficient to form a first precipitate and a first supernatant;
(b) isolating the first precipitate from the first supernatant;
(c) resuspending the first precipitate in a solution of ammonium sulfate to form a second precipitate and a second supernatant;
(d) separating the second supernatant from the second precipitate;
(e) increasing the concentration of ammonium sulfate in the second supernatant to form a third precipitate;
(f) collecting the third precipitate;
(g) forming a suspension of the third precipitate in dialysis buffer, the dialysis buffer comprising:
(i) a liquid carrier;
(ii) at least one stabilizer, wherein the stabilizer is selected from the group consisting of amylopectin, galatomannan polysaccharide, agarose, agaropectin, and gelatin; and
(iii) at least one additive selected from the group consisting of a zwitterionic buffer, an antibiotic agent, and sodium citrate; and
(h) dialyzing the suspension against dialysis buffer, wherein the suspension is held within a dialysis membrane capable of retaining the coagulation factor.

3. A factor concentrate prepared according to the method of claim 2.

4. A method of preparing a coagulation control, comprising the steps of:
(a) combining at least one stabilizer, a zwitterionic buffer and at least one coagulation factor in a liquid carrier to form a buffered solution, wherein the stabilizer is selected from the group consisting of amylopectin, galatomannan polysaccharide, agarose, agaropectin, and gelatin, the stabilizer being present in an amount sufficient to retard the degradation of the coagulation factor for at least one month when the solution is stored at 2° C. to 8° C.; and
(b) testing the buffered solution in a clotting time assay, and, if needed, adding to the solution the factor concentrate of claim 3 in an amount sufficient to adjust the clotting time assay within a desired range.

5. A coagulation control prepared according to the method of claim 4.

6. An assay for detecting clot formation comprising:
(a) combining a sample and a reagent, wherein the reagent is a composition of matter comprising a phospholipid coagulation factor in the liquid carrier that mediates a cascade pathway that leads to clot formation selected from the group consisting essentially of thromboplastin cephaloplastin, cephalin, and soy phosphatides; and at least one stabilizer for the phospholipid coagulation factor in the liquid carrier selected from the group consisting of amylopectin, galatomannan polysaccharide, agarose, agaropectin, and gelatin, the stabilizer being present in an amount sufficient to retard the degradation of the coagulation factor for at least one month when the composition of matter is stored at 2° C. to 8° C.;
(b) initiating clot formation when calcium ions are introduced with the sample and reagent; and
(c) determining a sample clotting time.

7. An assay according to claim 6, wherein the combining step (a) further comprises combining a factor deficient plasma with the sample and reagent, wherein the factor deficient plasma comprises:
(i) plasma selected from the group consisting of Factor II deficient, Factors II and VII deficient, Factors II, VII, and X deficient, Factor V deficient, Factor VII deficient, Factors VII and X deficient, Factor VIII deficient, Factor IX deficient, Factor X deficient, Factor XI deficient, Factor XII deficient, Factor XIII deficient, high molecular weight kininogen deficient, plasminogen activator inhibitor deficient, t-plasminogen activator deficient, prekallikrein deficient, protein C deficient, protein S, and protein X deficient plasmas; and
(ii) at least one stabilizer for the plasma, wherein the stabilizer is selected from the group consisting of amylopectin, galatomannan polysaccharide, agarose, agaropectin, and gelatin, the stabilizer being present in an amount sufficient to retard the degradation of the plasma for at least one month when the plasma is stored at 2° C. to 8° C.

8. An assay according to claim 6 further comprising:
(a) combining a control with a reagent, the control comprising;
(i) a liquid carrier;
(ii) a plurality of coagulation factors, wherein the coagulation factors are components in a cascade pathway that leads to clot formation; and
(iii) a stabilizer for the coagulation factors in the liquid carrier, wherein the stabilizer is selected from the group consisting of amylopectin, galatomannan polysaccharide, agarose, agaropectin, and gelatin, the stabilizer being present in an amount sufficient to retard the degradation of the coagulation factors for at least one month when the control is stored at 2° C. to 8° C.;
(b) initiating clot formation when calcium ions are introduced with the control and the reagent;
(c) determining a control clotting time; and
(d) relating the sample clotting time to the control clotting time to determine whether the sample clotting time falls within a select range.

9. An assay for detecting clot formation comprising:
(a) combining a sample and a reagent, wherein the reagent is selected from the group consisting of thromboplastin, cephaloplastin, cephalin, and soy phosphatides;
(b) initiating clot formation when calcium ions are introduced with the sample and reagent; and
(c) determining a sample clotting time;
(d) combining a control with a reagent, wherein the control is the coagulation control prepared by:
(i) combining at least one stabilizer, a zwitterionic buffer and at least one coagulation factor in a liquid carrier to form a buffered solution, wherein the stabilizer is selected from the group consisting of amylopectin, galatomannan polysaccharide, agarose, agaropectin, and gelatin and gum, the stabilizer being present in an amount sufficient to retard the degradation of the coagulation factor for at least one month when the solution is stored at 2° C. to 8° C.; and
(ii) testing the buffered solution in a clotting time assay, and, if needed, adding to the solution the factor concentrate of claim 3 in an amount sufficient to adjust the clotting time assay within a desired range;
(e) initiating clot formation when calcium ions are introduced with the control and the reagent;
(f) determining a control clotting time; and
(g) relating the sample clotting time to the control clotting time to determine whether the sample clotting time falls within a select range.

10. A test kit for use in an assay that detects clot formation, the test kit comprising:
(a) a first container comprising a reagent, wherein the reagent is a composition of matter comprising;
(b) combining a sample and a reagent, wherein the reagent is a composition of matter comprising aphospholipid coagulation factor in the liquid carrier that mediates a cascade pathway that leads to clot formation selected from the group consisting essentially of thromboplastin, cephaloplastin, cephalin, and soy phosphatides; and at least one stabilizer for the phospholipid coagulation factor in the liquid carrier selected from the group consisting of amylopectin, galatomannan polysaccharide, agarose, agaropectin, and gelatin, the stabilizer being present in an amount sufficient to retard the degradation of the coagulation factor for at least one month when the composition of matter is stored at 2° C. to 8° C.; and
(c) at least one other container comprising a control.

11. A test kit according to claim 10 wherein the control comprises:
(a) a liquid carrier;
(b) a plurality of coagulation factors in the liquid carrier, wherein the coagulation factors are components in a cascade pathway that leads to clot formation; and
(c) at least one stabilizer for the coagulation factors in the liquid carrier, wherein the stabilizer is selected from the group consisting of amylopectin, galatomannan polysaccharide, agarose, agaropectin, and gelatin, the stabilizer being present in an amount sufficient to retard the degradation of the coagulation factors for at least one month when the control is stored at 2° C. to 8° C.

12. A test kit for use in an assay that detects clot formation, the test kit comprising:
(a) a first container comprising a reagent, wherein the reagent is selected from the group consisting essentially of thromboplastin, cephaloplastin, cephalin, and soy phosphatides; and
(b) at least one other container comprising a control, wherein the control is the coagulation control of claim 5.

13. A method of replacing a coagulation factor in vivo comprising administering by intravenous infusion, an effective dose of a factor concentrate wherein the factor concentrate is prepared according to the method of claim 2
